(19) United States
(12) Patent Application Publication
(10) Pub. No.: US 2014/0120184 A1
(43) Pub. Date: May 1, 2014

(54) ARGON-BASED INHALABLE GASEOUS MEDICINAL PRODUCT FOR THE TREATMENT OF NEUROINTOXICATIONS

(71) Applicant: Air Liquide Sante (International), Paris (FR)

(72) Inventors: Marc LEMAIRE, Paris (FR); Jacques Abraini, Caen (FR)

(73) Assignee: Air Liquide Sante (International), Paris (FR)

(21) Appl. No.: 14/040,962

(22) Filed: Sep. 30, 2013

Related U.S. Application Data
(63) Continuation of application No. 12/969,190, filed on Dec. 15, 2010, now abandoned.
Application No. 12/969,190 is a continuation of application No. 11/836,536, filed on Aug. 9, 2007, now abandoned.
Application No. 11/836,536 is a division of application No. 11/007,684, filed on Dec. 8, 2004, now abandoned.

(30) Foreign Application Priority Data
Dec. 8, 2003 (FR) 0350997

(51) Int. Cl. A61K 33/00

(52) U.S. Cl. CPC A61K 33/00
USPC 424/718; 424/600

(57) ABSTRACT

The invention relates to the use of argon gas for producing all or part of an inhalable medicinal product intended to prevent or treat a neurointoxication in humans. The medicinal product contains argon in an effective proportion and acts on at least one brain receptor in order to regulate the functioning of dopamine-, glutamate-, serotonin-, acetylcholine-, taurine-, GABA- and/or noradrenalin-mediated neurotransmission systems. Preferably, the proportion by volume of argon in the gaseous medicinal product is between 15 and 80%. The neurointoxication is chosen from excitotoxicities engendering a state of addiction, acute cerebral accidents, neurodegenerative diseases, and psychiatric or neurological pathologies, in particular anxiety conditions, psychotic conditions, in particular schizophrenia, and epilepsy in its various forms.

The present application is a continuation of U.S. patent application Ser. No. 12/969,190, filed Dec. 15, 2010, which is a continuation of U.S. patent application Ser. No. 11/836,536, filed Aug. 9, 2007, which is a divisional of U.S. patent application Ser. No. 11/007,684, filed Dec. 8, 2004, which claims priority to French Patent Application No. 03 50997, filed Dec. 8, 2003, the entire contents of each of these applications incorporated herein.

### BACKGROUND

The invention relates to the use of argon for producing all or part of an inhalable medicinal product intended to treat or prevent a pathology having a neurotoxic effect, i.e. a neurointoxication.

In pathologies related to the neurotoxic effects of drugs generating an addiction, such as amphetamines, it is accepted that dopaminergic neurotransmission of nigrostriatal and mesolimbic origin participates in the psychostimulant and neurotoxic effects of these drugs.

However, recent studies by Del Arco et al., Neuropharmacology, 1999, vol. 38, p. 943-954, have shown that the facilitating effects of amphetamines are not limited to dopaminergic neurotransmission.

Thus, at the level of the striatum-nucleus accumbens complex, amphetamines induce not only an increase in dopamine release, but disturb serotonin, taurine, γ-aminobutyric acid (GABA), noradrenalin and glutamate neurotransmission systems.

Particularly advantageously, it has been shown that specific inhibition of glutamate transporters makes it possible to decrease both the hyperactivity (David et al. Neuropharmacology, 2001, vol. p. 409-411) and the increase in glutamate, but not of dopamine (Del Arco et al., Neuropharmacology, 1999, vol. 38, p. 943-954) subsequent to the injection of amphetamines, thus suggesting a determining role for glutamate in the psychostimulant effects of amphetamines.

Moreover, recent studies, carried out in vitro, have shown that xenon and nitrous oxide (N₂O) can behave as N-methyl-D-aspartate (NMDA) glutamate receptor antagonists; Franks et al., Nature, 1998, vol. 396, p. 324; Jevtovic-Todorovic et al., Nature Med., 1998, vol. 4, p. 460-463.

In addition, in the context of the study of the endogenous hyperalgesic opioid system in the negative placebo response, F. J. Lichtigfeld and M. A. Gillman, Intern. J. Neuroscience, 1989, vol. 49, p. 71-74, conclude that nitrous oxide has a slightly better effect on alcohol withdrawal than the placebo, although, for more than 50% of the individuals, an identical positive effect was also observed with the placebo.

However, the same authors add, in Nitrous Oxide and the Aws, p. 785, that the beneficial effect of nitrous oxide depends strictly on its concentration since anaesthetic or pre-anaesthetic concentrations are ineffective, or even counterproductive in certain cases, an analgesic concentration being recommended.

In Postgrad. Med. J. Clinical Toxicology, 1990, vol. 66, p. 543-546, the same authors explain that the concentrations of nitrous oxide can range from less than 15% to more than 70% according to the individuals, as a function of their degree of alcohol dependency.

Moreover, document EP-A-1158992 teaches the use of xenon or of a mixture of xenon with oxygen, nitrogen or air for treating neurointoxications.

However, the use of xenon or the mixtures described by that document is not entirely satisfactory in practice, in particular due to the appearance of toxicity for certain xenon contents and given the high cost of this compound; David et al., J. Cereb. Blood Flow. Metab., 2003, vol. 23, p. 1168-1173.

The present invention falls within this context and is aimed at improving the existing inhalable medicinal products intended to effectively prevent or treat a neurointoxication in humans which is characterized by a cerebral dysfunction of one or more neurotransmission systems.

### SUMMARY

The solution of the invention relates then to the use of argon (Ar) gas for producing all or part of an inhalable medicinal product intended to prevent or treat a neurointoxication in humans.

### BRIEF DESCRIPTION OF THE DRAWINGS

For a further understanding of the nature and objects for the present invention, reference should be made to the following detailed description, taken in conjunction with the accompanying drawings, in which like elements are given the same or analogous reference numbers and wherein:

FIG. 1 illustrates the effects on locomotor activity; and

FIG. 2 illustrates the production of stereotypic movements.

### DESCRIPTION OF PREFERRED EMBODIMENTS

The term “neurointoxication” is intended to mean a condition, a disorder or a pathology of the central nervous system, the etiopathogenesis of which involves, at least partly, an excitotoxic process, in particular a dysfunction of glutamate-mediated excitatory neurotransmission; see in particular the document Parsons et al., Drug News Perspect., 1998, vol. 11, pages 523-569.

Consequently, treatment in particular of the following falls within the context of the present invention:
neurotoxic effects of drugs or other substances which can generate an addiction, such as amphetamines and amphetamine derivatives, opiate substances and their derivatives, cocaine and its derivatives, tobacco, cannabis and/or alcohol;
acute cerebral accidents such as cranial traumas and strokes (including cerebral ischemia);
neurodegenerative diseases such as Alzheimer's disease, Parkinson's disease, Huntington's disease (chorea), amyotrophic lateral sclerosis, acute disseminated encepholomyelitis, late dyskinesia, and olivopontocerebellar degeneration; and
various psychiatric or neurological pathologies such as anxiety conditions, psychotic conditions, in particular schizophrenia, and epilepsy in its various forms.

According to the case, the use of the invention may comprise one or more of the following technical characteristics:
the neurointoxication results from a cerebral dysfunction, i.e. an excess of or a decrease in one or more neurotransmitter systems;
the mixture containing the argon in an effective proportion acts on at least one brain receptor in order to regulate the functioning of dopamine-, glutamate-, serotonin-, taurine-, acetylcholine-, GABA- and/or noradrenalin-mediated neurotransmission systems;
the proportion by volume of argon in said gaseous mixture is between 15 and 80%;
the proportion by volume of argon is between 30 and 75%;
the argon is in a gaseous mixture with at least one additional gaseous compound chosen from xenon, krypton and nitrous oxide (N₂O);
the gaseous medicinal product also contains oxygen, nitrogen or mixtures thereof, in particular air;
the gaseous mixture is a binary mixture consisting of argon and of oxygen for the remainder; the gaseous medicinal product is preferably ready to use, i.e. it can be administered to the patient directly without undergoing any predilution;
the neurointoxication is chosen from conditions, disorders or pathologies of the central nervous system, the etiopathogenesis of which involves, at least partly, an excitotoxic process, such as the neurotoxic effects of drugs or of substances which can generate a state of addiction, acute cerebral accidents, neurodegenerative diseases, and various psychiatric or neurological pathologies. The expression “neurointoxication engendering a state of addiction” is intended to mean a condition, a disorder or a pathology related to the neurotoxic effects of a drug, molecule or substance generating an addiction or a habit in humans or animals. The substance, drug or molecule generating the addiction is chosen from the group made up of amphetamines and their derivatives, opiate substances and their derivatives, cocaine and its derivatives, tobacco, alcohol and cannabis, or any other similar or analogous drug. The term “acute cerebral accident” is intended to mean a condition, a disorder or a pathology subsequent to a violent and sudden event of exogenous or endogenous origin. The exogenous event may be a cranial trauma, whereas the endogenous event may be the rupture or the occlusion of an artery or of a cerebral blood vessel. The term “neurodegenerative disease” is intended to mean a condition, a disorder or a pathology related to the degeneration and the death of certain cerebral neurons;
the inhalable medicinal product is packaged at a pressure of 2 bar to 350 bar, preferably between 2 bar and 200 bar.

The invention also relates to a gaseous mixture containing argon as an inhalable medicinal product, for treating a neurointoxication in humans.

According to the case, the gaseous mixture of the invention may comprise one or more of the following technical characteristics:
it is made up of argon and of oxygen;
it is made up of 20 to 80% by volume of argon and of oxygen for the remainder, preferably of 30 to 75% of argon. In all cases, the proportion of argon and/or of oxygen in the gaseous mixture may be adjusted according to the duration of the treatment.

In other words, the idea on which the present invention is based is therefore that the agonist properties of argon on GABA A receptors mediating inhibitory neurotransmission (see Abraini et al., Anesth Analg, 2003, vol. 96, p. 746-749, 2003) can be used, by virtue of their inhibitory nature, to limit the excitatory effects of glutamate in order to prevent and/or treat neurointoxications, in particular the neurotoxic effects of drugs or substances generating an addiction, such as amphetamines and their derivatives, opiate substances and their derivatives, cocaine and its derivatives, tobacco, alcohol, cannabis and any other substances engendering a dependency.

In general, the gaseous medicinal product according to the invention can be administered to the patient via his or her upper airways, i.e. by inhalation via his or her nose and/or mouth, by means of a patient respiratory interface, such as a breathing mask or a tracheal tube, or one or more supply tubes serving to convey the gaseous medicinal product from a source containing said medicinal product to the interface, and a medical ventilator used to send the gas and/or to withdraw the gas from the patient.

### EXAMPLE

Evaluation of the neuroprotective potential of argon gas, administered alone or as a mixture with nitrous oxide, on the development and expression of sensitization to D-amphetamine.

The aim of the study is to evaluate the neuroprotective potential, on sensitization to D-amphetamine, of argon, for which the mechanisms of action, that are still poorly understood, could involve an agonist action with respect to GABA_{A} receptors, in particular with respect to the benzodiazepine site, the argon being administered alone or as a mixture; Abraini et al., Anesth Analg, 2003, vol. 96, p. 746-749, 2003.

To do this, adult male Sprague-Dawley rats that weighed approximately 220 g when they arrived in the laboratory were used. Throughout the study, the animals were placed under standard animalhouse conditions, in groups of 8, so as to avoid the appearance of a stress reaction subsequent to isolation. They had water and food ad libitum.

The D-amphetamine sensitization protocol followed and the trials for treatment by administration of gas used were as follows:

For 3 consecutive days (from D1 to D3), 8 groups of animals (8 rats per group) were administered intraperitoneally (i.p.) either D-amphetamine (amph: 1 mg/ml/kg), or a saline solution (saline: 1 ml/kg) for the control animals.

After each injection of D-amphetamine, the rats were immediately placed, for 3 hours, in a closed chamber having a volume of 100 litres that was swept under dynamic conditions, namely:
with air (group 1: saline; group 2: amph);
with a mixture of argon at 37.5 vol % and of nitrous oxide at 37.5 vol % (group 3: saline; group 4: amph), the remainder being oxygen;
with a mixture of argon at 50 vol % and of nitrous oxide at 25 vol % (group 5: saline; group 6: amph), the remainder being oxygen;
with argon at 75 vol % (group 7: saline; group 8: amph), the remainder being oxygen.

The gases used in this study were administered under dynamic conditions at an initial rate of 10 l.min⁻¹ for 30 minutes, and then at a constant rate of 1 l.min⁻¹ for 2 h 30 min.

By proceeding in this way, the effective concentration after treatment for 30 minutes is equal to 95% of the desired final concentration (corresponding to the mixture used) and the cumulative dose×time value is more than 25% greater than the dose×time value obtained using, as previously, a constant rate of introduction of gases of 5 l.min⁻¹; see the document Abraini and David, for Air Liquide Santé International “étude du potentiel neuroprotecteur du xénon et du protoxyde d'azote” [study of the neuroprotective potential of xenon and of nitrous oxide], May 2001-October 2003), which makes it possible to optimize the treatment in its initial phase; the total cumulative dose×time values are substantially equal; see Table 1 below.

TABLE 1

| 1-% | | Tps | | cumulative | Tps | | cumulative |
|---|---|---|---|---|---|---|---|
| [final] | Kc | (100/10) | D * T | D * T | (100/5) | D * T | D * T |
| 1 | | | | | 0 | | |
| 0.95 | 0.051 | 0.5 | 0.6 | 0.6 | 1.0 | 1.28 | 1.28 |
| 0.9 | 0.105 | 1.1 | 2.0 | 2.6 | 2.1 | 4.06 | 5.34 |
| 0.85 | 0.163 | 1.6 | 3.6 | 6.2 | 3.3 | 7.14 | 12.48 |
| 0.8 | 0.223 | 2.2 | 5.3 | 11.5 | 4.5 | 10.61 | 23.09 |
| 0.75 | 0.288 | 2.9 | 7.3 | 18.8 | 5.8 | 14.52 | 37.61 |
| 0.7 | 0.357 | 3.6 | 9.5 | 28.3 | 7.1 | 18.97 | 56.58 |
| 0.65 | 0.431 | 4.3 | 12.0 | 40.3 | 8.6 | 24.09 | 80.67 |
| 0.6 | 0.511 | 5.1 | 15.0 | 55.3 | 10.2 | 30.02 | 110.68 |
| 0.55 | 0.598 | 6.0 | 18.5 | 73.8 | 12.0 | 36.98 | 147.66 |
| 0.5 | 0.693 | 6.9 | 22.6 | 96.4 | 13.9 | 45.27 | 192.94 |
| 0.45 | 0.799 | 8.0 | 27.7 | 124.1 | 16.0 | 55.31 | 248.25 |
| 0.4 | 0.916 | 9.2 | 33.9 | 157.9 | 18.3 | 67.73 | 315.98 |
| 0.35 | 1.050 | 10.5 | 41.7 | 199.7 | 21.0 | 83.46 | 399.43 |
| 0.3 | 1.204 | 12.0 | 52.0 | 251.7 | 24.1 | 104.05 | 503.48 |
| 0.25 | 1.386 | 13.9 | 66.1 | 317.8 | 27.7 | 132.18 | 635.67 |
| 0.2 | 1.609 | 16.1 | 86.5 | 404.3 | 32.2 | 172.94 | 808.60 |
| 0.15 | 1.897 | 19.0 | 118.7 | 522.9 | 37.9 | 237.34 | 1045.94 |
| 0.1 | 2.303 | 23.0 | 177.4 | 700.3 | 46.1 | 354.78 | 1400.72 |
| 0.05 | 2.996 | 30.0 | 320.6 | 1020.9 | 59.9 | 641.16 | 2041.89 |
| 0.01 | 4.605 | 160.9 | 6352.8 | 7373.7 | 92.1 | 1561.15 | 3603.04 |
| 0.01 | 4.605 | 180 | 943.3 | 8317.0 | 180 | 4350.88 | 7953.92 |

Table 1 indicates the cumulative doses (dose×time; cumulative D*T) as a function of the dynamic sweep conditions used (corresponding to the rate of introduction of the gases or mixtures of gases) to saturate a chamber having a volume of 100 litres. It is noted that, after 30 minutes, the cumulative dose obtained using an initial rate of 10 l.min⁻¹ (followed by a constant rate of 1 l.min⁻¹ for 2 h 30 min) is approximately 25% greater than the cumulative dose obtained using a constant rate of 5 l.min⁻¹.

The locomotor activity and the righting activity of the animals were evaluated at D6, after an i.p. injection of a saline solution (1 ml/kg) in order to determine the actual effects of the treatments administered with the various gases and mixtures of gas, and on D7 after an i.p. administration of D-amphetamine (1 mg/ml/kg) in order to evaluate the effects of the gases and mixtures of gas on sensitization to D-amphetamine. The locomotor activity and the stereotypic righting activity of the animals in response to these injections were registered by means of a photoelectric cell actimetry system (Imêtronic, Pessac, France).

The D-amphetamine (D-amphetamine sulphate, ref. A5880) was obtained from Sigma-Aldrich (Illkirch, France).

The medical air, the argon at 75 vol %, and the mixture of nitrous oxide at 50 vol % and of argon at 75 vol %, the remainder being oxygen, were provided by Air Liquide Santé International (Paris, France).

The mixture of nitrous oxide at 37.5 vol % and of argon at 37.5 vol %, the remainder being oxygen, was prepared from nitrous oxide at 75 vol % and argon at 75 vol %, provided by Air Liquide Santé International, using calibrated flow meters also provided by Air Liquide Santé International.

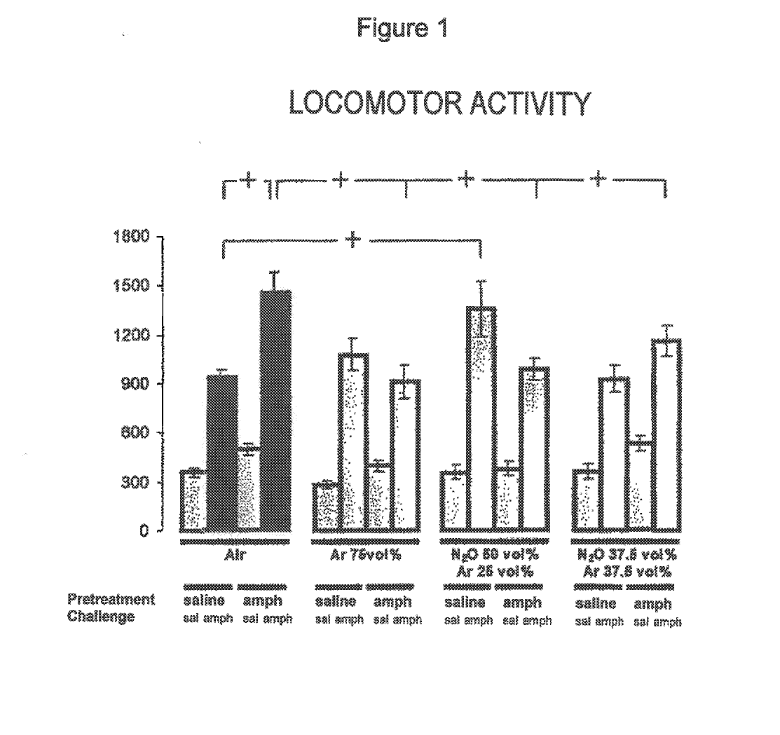

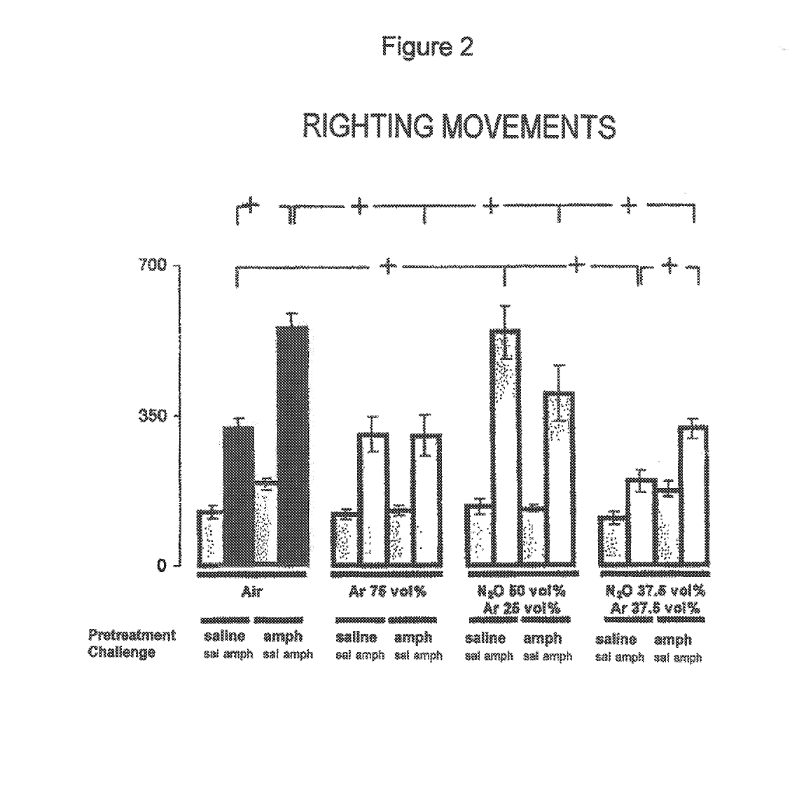

The results obtained (see FIGS. 1 and 2) are expressed as the mean±the standard error of the mean. The comparison of the groups was carried out by means of nonparametric tests: Kruskall-Wallis analysis of the variants, completed, in the event of a significant result, by means of Mann-Whitney U test.

The left-hand sections of FIGS. 1 and 2 illustrate the process of sensitization induced by the repeated administration of D-amphetamine.

More precisely, FIG. 1 illustrates the effects, at D7, on locomotor activity induced by the repeated injection of D-amphetamine, whereas FIG. 2 illustrates the production of stereotypic movements, i.e. righting movements, induced by the repeated administration of D-amphetamine (1 mg/kg).

The challenge with D-amphetamine engenders an increase in the locomotor activity and also in the stereotypic movements, such that the locomotor activity and the stereotypic movements (i.e. the righting movements) of the animals pretreated with D-amphetamine appear to be significantly greater than those of the control rats pretreated by means of a saline solution, in the test with D-amphetamine carried out on D7 (P<0.05).

FIGS. 1 and 2 illustrate the effects, on the locomotor activity and the righting movements induced by the repeated administration of D-amphetamine, of a treatment by means of argon at 75 vol %, of a mixture of nitrous oxide at 50 vol % and of argon at 25 vol %, or of a mixture of nitrous oxide at 37.5 vol % and of argon at 37.5 vol % (the remainder being oxygen).

The exposure, immediately after administration of D-amphetamine, to argon at 75 vol % or to a mixture of nitrous oxide at 50 vol % and of argon at 25 vol % or to a mixture of nitrous oxide at 37.5 vol % and of argon at 37.5 vol % induces blocking of the development of the locomotor activity corresponding to the process of sensitization to D-amphetamine.

The locomotor activity obtained on D7 during the challenge with D-amphetamine in the rats sensitized for 3 days to D-amphetamine and treated with (i) argon at 75 vol %, or (ii) a mixture of nitrous oxide at 50 vol % and of argon at 25 vol %, or (iii) a mixture of nitrous oxide at 37.5 vol % and of argon at 37.5 vol %, is less than that of the rats sensitized to D-amphetamine and treated with air, but not significantly different from the locomotor activity of the animals which received, for 3 days, a saline solution and (i) argon at 75 vol %, or (ii) a mixture of nitrous oxide at 50 vol % and of argon at 25 vol %, or (iii) a mixture of nitrous oxide at 37.5 vol % and of argon at 37.5 vol % (corresponding to an acute injection of D-amphetamine).

These results show a total inhibitory effect of argon at 75 vol % (P<0.005), of the mixture of nitrous oxide at 50 vol % and of argon at 25 vol % (P<0.002) and, to a lesser degree, of the mixture of nitrous oxide at 37.5 vol % and of argon at 37.5 vol % (P<0.05), on the locomotor hyperactivity inherent to the development of sensitization to D-amphetamine.

However, it should be noted, in the rats that received a saline solution for 3 days and were exposed to the mixture of nitrous oxide at 50 vol % and of argon at 25 vol %, that the locomotor activity engendered by the challenge with D-amphetamine, carried out on D7, is significantly greater (P<0.01) than the locomotor activity measured during the same challenge in the animals that received a saline solution for 3 days and were exposed to air, the level of locomotor activity reached being comparable to the locomotor activity generally recorded after sensitization.

This result could reflect a potential neurotoxic effect of the mixture of nitrous oxide at 50 vol % and of argon at 25 vol %, similar to the effects already known for xenon at 75 vol %; see Abraini and David, for Air Liquide Santé International “Etude du potentiel neuroprotecteur du xénon et du protoxyde d'azote” [Study of the neuroprotective potential of xenon and of nitrous oxide], May 2001-October 2003.

Moreover, exposure to argon at 75 vol %, to the mixture of nitrous oxide at 50 vol % and of argon at 25 vol % or to the mixture of nitrous oxide at 37.5 vol % and of argon at 37.5 vol %, immediately after injection of D-amphetamine, induces blocking of the development of the righting activity inherent to the process of sensitization to D-amphetamine.

The stereotypic righting activity obtained at D7 during the challenge with D-amphetamine in the rats sensitized for 3 days to D-amphetamine and treated with (i) argon at 75 vol %, or (ii) a mixture of nitrous oxide at 50 vol % and of argon at 25 vol %, or (iii) a mixture of nitrous oxide at 37.5 vol % and of argon at 37.5 vol %, is less than that of the rats sensitized to D-amphetamine for 3 days and treated with air, but not significantly different from the righting activity of the animals that received a saline solution for 3 days and (i) argon at 75 vol %, or (ii) a mixture of nitrous oxide at 50 vol % and of argon at 25 vol %, or (iii) a mixture of nitrous oxide at 37.5 vol % and of argon at 37.5 vol % (corresponding to an acute injection of D-amphetamine).

These results reflect an inhibitory effect of argon at 75 vol % (P<0.005), of the mixture of nitrous oxide at 37.5 vol % and of argon at 37.5 vol % (P<0.001) and, to a lesser degree, of the mixture of nitrous oxide at 50 vol % and of argon at 25 vol % (P<0.02) on the righting activity corresponding to the development of the sensitization to D-amphetamine.

However, it should be noted here again, in the rats that received a saline solution for 3 days and were exposed to the mixture of nitrous oxide at 50 vol % and of argon at 25 vol %, that the righting activity engendered by the challenge with D-amphetamine carried out at D7 appears to be significantly greater (P<0.002) than the righting activity measured during the same challenge in the animals that received a saline solution for 3 days and were exposed to air, the level of stereotypic activity reached being comparable to the righting activity generally registered after sensitization.

This result, which corroborates the data obtained above for the locomotor activity, supports the idea of a potentially neurotoxic effect of the mixture of nitrous oxide at 50 vol % and of argon at 25 vol %.

In fact, although the mixture of nitrous oxide at 50 vol % and of argon at 25 vol % shows an inhibitory effect on the locomotor activity and the righting activity induced by the repeated administration of D-amphetamine, the results obtained suggest a possible neurotoxic effect of this mixture.

It therefore appears that the other gases and mixtures of gases tested, i.e. argon at 75 vol % and the mixture of nitrous oxide at 37.5 vol % and of argon at 37.5 vol %, are preferred in the context of the present invention since they unquestionably make it possible to block both the locomotor hyperactivity and the stereotypic righting activity inherent to the development of D-amphetamine sensitization, without any neurotoxic effect being observed.

In other words, all the results obtained show that argon, optionally with nitrous oxide added to it, has inhibitory effects on the development of D-amphetamine sensitization.

More precisely, as regards argon, it should be emphasized particularly advantageously, beyond the quantitative data which attest that this gas has an unquestionable inhibitory effect at 75 vol %, that the results obtained from a qualitative point of view appear to be very surprising.

Thus, from the 2nd day of treatment, i.e. after one day of sensitization to D-amphetamine and of treatment with argon at 75 vol %, the animals were found to be astonishingly calm and “cooperative” throughout the remainder of the experimental protocol, including during the intraperitoneal injections of D-amphetamine.

This subjective piece of data, that is nevertheless of great interest, could reflect a mechanism of action that is radically different from that of nitrous oxide, the antagonist properties of which with respect to NMDA glutamate receptors are currently well identified; see in particular Jevtovic-Todorovic et al., et 1998; Yamakura et al., 2000.

The mode of action of argon still remains very largely unknown, despite a recent neuropharmacological study which suggested that argon could exhibit GABA_{A} receptor agonist properties; Abraini et al., Anesth Analg, 2003, vol. 96, p. 746-749, 2003.

Moreover, the combination of nitrous oxide at 37.5 vol % and of argon at 37.5 vol % also shows an inhibitory effect on the development of the process of D-amphetamine sensitization.

This effect obtained with percentages of nitrous oxide and of argon that are respectively relatively low could indicate that these two gases have additive or synergistic properties.

In this sense, it should be noted that the combination of nitrous oxide at 50 vol % and of argon at 25 vol % could exhibit potentially neurotoxic properties and, as a result, the use of nitrous oxide and of argon in combination should be carried out with care, i.e. by choosing the respective proportions of these compounds carefully.

All these results make it possible to envisage a potential therapeutic value for argon, alone or as a mixture with nitrous oxide (N₂O), in particular for the treatment of addiction to psychostimulant substances or, at the very least, to amphetamines.

According to the invention, the gaseous medicinal product is a binary gaseous mixture consisting of argon and of oxygen for the remainder, or a ternary mixture consisting of argon, of nitrogen and of oxygen; the gaseous medicinal product is preferably ready to use.

In particular, the gaseous mixture is made up of 20 to 80% by volume of argon, and of nitrogen and oxygen for the remainder, preferably of 30 to 75% of argon.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

What is claimed is:

1. An inhalable medical gaseous composition for administration to humans that is suitable for treating amphetamine addiction, said composition comprising from 15% to 80% by volume of gaseous argon with the remainder being oxygen or nitrogen or mixtures thereof.

2. The inhalable medical gaseous composition of claim 1, wherein the argon is present in an amount from 30 to 75% by volume.

3. The inhalable medical gaseous composition of claim 1, wherein the composition comprises from 20% to 80% by volume argon with the remainder being oxygen.

4. The inhalable medical gaseous composition of claim 1, wherein the composition comprises from 20% to 80% by volume argon with the remainder being oxygen and nitrogen.

5. An inhalable medical gaseous composition comprising from 15% to 80% by volume of gaseous argon and at least on additional gaseous compound selected from xenon, krypton and nitrous oxide.

6. The inhalable medical gaseous composition of claim 1, wherein the argon is present in an amount from 30 to 75% by volume.

7. The inhalable medical gaseous composition of claim 5, wherein the composition also comprises oxygen or nitrogen or mixtures thereof.

8. The inhalable medical gaseous composition of claim 7, wherein the composition comprises 15% to 80% by volume of gaseous argon with the remainder being oxygen and at least on additional gaseous compound selected from xenon, krypton and nitrous oxide.

9. The inhalable medical gaseous composition of claim 7, wherein the composition comprises 15% to 80% by volume of gaseous argon with the remainder being oxygen and nitrogen and at least on additional gaseous compound selected from xenon, krypton and nitrous oxide.

10. A method for treating neurointoxication in a human, the method comprising administering by inhalation to a human a therapeutically effective amount of an inhalable medicinal product that comprises from between 15% and 80% by volume of gaseous argon.

11. The method of claim 10, wherein the neurointoxication results from a cerebral dysfunction of one or more neurotransmitter systems.

12. The method of claim 10, wherein said inhalable medicinal product acts on at least one brain receptor in order to regulate the functioning of at least one mediated neurotransmission system selected from the group consisting of:
a) dopamine-mediated neurotransmission system,
b) glutamate-mediated neurotransmission system,
c) serotonin-mediated neurotransmission system,
d) acetylcholine-mediated neurotransmission system,
e) taurine-mediated neurotransmission system,
f) GABA-mediated neurotransmission system, and
g) Noradrenalin-mediated neurotransmission system.

13. The method of claim 10, wherein the effective proportion of argon in said inhalable medicinal product is between about 30% and about 75% by volume.

14. The method of claim 10, wherein said argon containing gas comprises at least one additional gaseous compound selected from the group consisting of xenon, krypton and nitrous oxide.

15. The method of claim 14, wherein the inhalable medicinal product further comprises at least one additional gaseous compound selected from the group consisting of oxygen, nitrogen, or mixtures thereof.

16. The method of claim 10, wherein the inhalable medicinal product further comprises at least one additional gaseous compound selected from the group consisting of oxygen, nitrogen, or mixtures thereof.

17. The method of claim 10, wherein the inhalable medicinal product is a binary gaseous mixture comprising argon with the remainder being oxygen.

18. The method of claim 10, wherein said inhalable medicinal product is a ternary gaseous mixture comprising argon with the remainder being nitrogen and oxygen.

19. The method of claim 10, wherein the neurointoxication is selected from the group consisting of:
a) excitotoxicities engendering a state of addiction,
b) acute cerebral accidents,
c) neurodegenerative diseases,
d) psychiatric pathologies,
e) neurological pathologies,
f) psychotic conditions, and
g) epilepsy in its various forms.

20. The method of claim 19, wherein the neurointoxication is an excitotoxicities engendering a state of addiction.

21. The method of claim 20, wherein the addition is to amphetamines.
